(19) United States
(12) Patent Application Publication
(10) Pub. No.: US 2024/0139068 A1
(43) Pub. Date: May 2, 2024

(54) STORAGE AND CARRYING DEVICE FOR EFFICIENT MANAGEMENT OF SINGLE-USE EYE DROP VIALS

(71) Applicant: Mary Krygiel, Ashburn, VA (US)

(72) Inventor: Mary Krygiel, Ashburn, VA (US)

(21) Appl. No.: 17/973,888

(22) Filed: Oct. 26, 2022

(51) Int. Cl. A61J 1/16; A61J 1/14

(52) U.S. Cl. CPC A61J 1/16; A61J 1/1412

(57) ABSTRACT

Apparatus for management of single-use eye drop vials sealed with twist-off tabs. The apparatus includes first and second new vial storage compartments, a trash storage compartment for used vials and separated twist-off tabs, and an opened-vial storage compartment for holding an eye drop vial while it is in use throughout a day. The first and second new vial storage compartments can be used for storing two different types of eye drops. Each storage compartment can have a separate latching lid. The first and second new vial storage compartments each include a bottom panel having a set of slots, each slot receiving a twist-off tab of a new vial when the new vial is inserted therein. The opened-vial storage compartment is made to appear distinct from both the first and second new vial storage compartments. The apparatus helps avoid wasting eye drops, while keeping new, partially used, and used vials separate.

### FIELD OF THE INVENTION

The invention relates generally to single-use eye drop vials, and particularly to devices for storage and carrying of single-use eye drop vials.

### BACKGROUND OF THE INVENTION

When one suffers from moderate to severe dry eye, one often must use multiple vials per day of single-use preservative-free eye drops. After use, these vials and their separated twist-off tabs end up on the bottom of purses, pockets, and/or car floors, for example.

The twist-off tabs are clear, small, and hard to see when they fall onto the floor. Consequently, the twist-off tabs are easily stepped on by bare feet when they fall onto bathroom floors.

Also, without a device to track one's portable inventory of vials, one may not have enough vials available on your person for the day.

And if one uses more than one type of single-use eye drops (e.g., gel-based eye drops and oil-based eye drops), it can be difficult to quickly and easily tell how many vials of each that you have on hand for the day.

Counter-top devices are currently on the market that hold multiple unopened vials for use. However, such devices are not portable, and present other problems.

### SUMMARY OF THE INVENTION

A general aspect of the invention is an apparatus for management of single-use eye drop vials, each single-use eye drop vial being sealed with a twist-off tab. The apparatus includes: a first new vial storage compartment configured to store a first plurality of new vials, the first new vial storage compartment having a top lid, and a bottom panel having a plurality of slots, each slot configured to receive a twist-off tab of a new vial when the new vial is inserted therein; a second new vial storage compartment configured to store a second plurality of new vials, the second new vial storage compartment having a top lid, and a bottom panel having a plurality of slots, each slot configured to receive a twist-off tab of a new vial when the new vial is inserted therein; and a trash storage compartment located below both the first and second storage compartments, the trash storage compartment configured to receive a plurality of twist-off tabs and a plurality of used vials, the trash storage compartment having a top lid for receiving used vials, and a bottom lid for emptying the trash storage compartment.

In some embodiments, the apparatus further includes: an opened-vial storage compartment configured to store a single opened partially used vial, the single opened partially used vial for use within 24 hours of opening.

In some embodiments, the opened-vial storage compartment has a separately operable latching lid.

In some embodiments, the opened-vial storage compartment is configured to appear distinct from both the first and second new vial storage compartments.

In some embodiments, the opened-vial storage compartment is configured to appear distinct from both the first and second new vial storage compartments by imparting a different color to the opened-vial storage compartment.

In some embodiments, the first new vial storage compartment and the second new vial storage compartment are configured to appear distinct such that a user can separately store two different types of eye drops, one type in each of the first and second new vial storage compartments.

In some embodiments, the first and second new vial storage compartments are configured to appear distinct by imparting a different color to each new vial storage compartment.

In some embodiments, each lid is configured to be latched open and latched closed.

In some embodiments, the first storage compartment top lid, and the second storage compartment top lid are continuous.

In some embodiments, the first storage compartment top lid, the second storage compartment top lid, and the used vial storage compartment top lid are all continuous with each other.

In some embodiments, the first new vial storage compartment is configured to store a first plurality of new vials in an inverted vertical orientation, each new vial having a twist-off tab underneath the new vial.

In some embodiments, the trash storage compartment is configured to receive a plurality of used vials either vertically or horizontally.

In some embodiments, the apparatus is made from a material that is light-weight so as to enhance portability.

In some embodiments, the bottom panel has a plurality of slots, each slot configured to receive a twist-off tab of a new vial when the new vial is inserted therein, and each slot is configured to resist turning of an inserted twist-off tab, while also allowing the twist-off tab once separated from the new vial to pass down through the slot into the trash storage compartment.

In some embodiments, the trash storage compartment has a side-opening lid that is latched.

In some embodiments, the trash storage compartment has a bottom-opening lid that is latched.

In some embodiments, the trash storage compartment includes a divider configured to separate the twist-off tabs and the used vials.

In some embodiments, the trash storage compartment has two independently openable lids, a first lid for inserting used vials and twist-off tabs, and a second lid for removing used vials and twist-off tabs.

Another general aspect of the invention is an apparatus for management of single-use eye drop vials, each single-use eye drop vial being sealed with a twist-off tab, this apparatus including: a first new vial storage compartment configured to store a first plurality of new vials, the first new vial storage compartment having a top lid, and a bottom panel having a plurality of slots, each slot configured to receive a twist-off tab of a new vial when the new vial is inserted therein; a second new vial storage compartment, adjacent to the first new vial storage compartment, the second new vial storage compartment configured to store a second plurality of new vials, the second new vial storage compartment having a top lid, and a bottom panel having a plurality of slots, each slot configured to receive a twist-off tab of a new vial when the new vial is inserted therein; an opened-vial storage compartment, adjacent to at least one of the first new vial storage compartment and the second new vial storage compartment, the opened-vial storage compartment configured to store a single opened partially used vial, the single opened partially used vial for use within 24 hours of opening; and a trash storage compartment located below both the first and second storage compartments, the trash storage compartment configured to receive a plurality of twist-off tabs and a plurality of used vials, the trash storage compartment having a top lid for receiving used vials, and a bottom lid for emptying the trash storage compartment.

In some embodiments, the opened-vial storage compartment has a separately operable latching lid, and the opened-vial storage compartment is configured to appear distinct from both the first new vial storage compartment and the second new vial storage compartment.

### BRIEF DESCRIPTION OF THE DRAWINGS

Many additional features and advantages will become apparent to those skilled in the art upon reading the following description, when considered in conjunction with the accompanying drawings, wherein:

FIG. 1 is a front view of an embodiment of the apparatus for management of single-use eye drop vials, showing the New Vial lid of the New Vial Compartment, the Current Vial lid of the Current Vial Compartment, and the Trash Chute lid of the Waste Compartment, all three lids in a closed state.

FIG. 2 is a front view of the embodiment of FIG. 1, showing the New Vial lid in an open state, revealing a set of new vials in the New Vial Compartment, showing the Current Vial lid in a partly open state, and an empty Current Vial Compartment, and showing the Trash Chute lid in a partly closed state.

FIG. 3 is a front view of the embodiment of FIG. 2, showing a new vial missing from the set of new vials in the New Vial Compartment, and showing a partly used vial entering the Current Vial Compartment.

FIG. 4 is a front view of the embodiment of FIG. 1, showing the Current Vial lid in a closed state, and showing the partly used vial now as an empty vial entering the Trash Chute.

FIG. 5 is an isometric view of the embodiment of FIG. 2, showing a set of new vials in the New Vial Compartment, showing Current Vial Compartment without a partly used vial and with the Current Vial lid in a partly open state, showing the top Trash Chute lid in an open state, also showing the side Waste Lid of the Waste Compartment in a closed state.

FIG. 6 is a front view of the embodiment of FIG. 5, showing the top Trash Chute lid in a partly open state, and showing the side Waste Lid of the Waste Compartment in a partly open state.

FIG. 7 is an isometric view of the embodiment of FIG. 6, showing the top Trash Chute lid in an open state, and showing the side Waste Lid of the Waste Compartment in a fully open state, revealing a fully used vial along with its detached tab resting on the floor of the Waste Compartment.

FIG. 8 is a bottom front perspective view of the embodiment of FIG. 7, showing each tab of a full set of new vials inserted into a respective slot in the floor of the New Vial Compartment, also showing the side Waste Lid of the Waste Compartment in a fully closed state, and showing the bottom opening of the Trash Chute that empties into the Waste Compartment.

### DETAILED DESCRIPTION

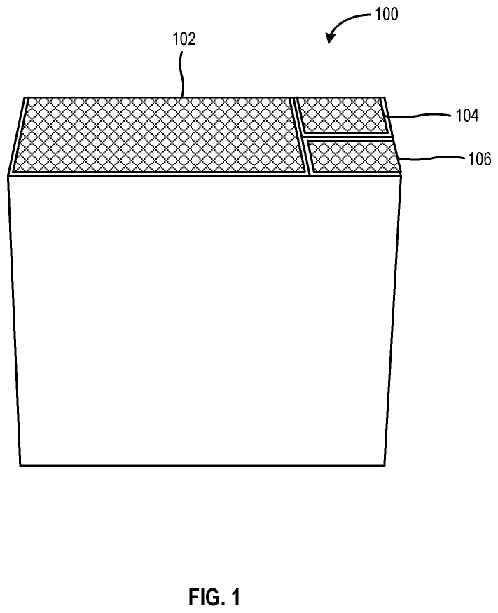

With reference to FIG. 1, an embodiment 100 of the apparatus for management of single-use eye drop vials is shown. This embodiment 100 includes a New Vial Compartment covered by a New Vial lid 102, a Current Vial Compartment covered by a Current Vial lid 104, and a Waste Compartment covered by a Trash Chute lid 106. The three lids 102, 104, and 106 are all shown in a closed state.

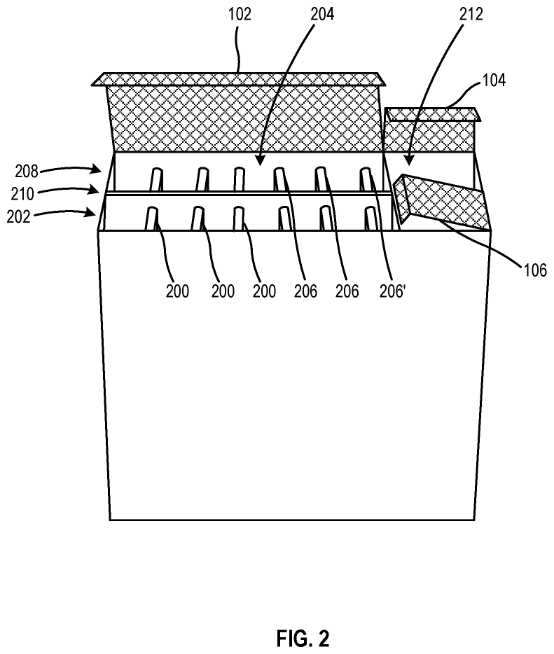

Referring to FIG. 2, the embodiment of FIG. 1 is shown, wherein the New Vial lid 102 is in an open state, revealing a first set of new vials 200 in a first half 202 of the New Vial Compartment 204. A second set of vials 206 are in a second half 208 of the New Vial Compartment 204. The first half 202 and the second half 208 of the New Vial Compartment 204 are separated by a dividing wall 210.

Also, the Current Vial lid 104 is shown in a partly open state, revealing an empty Current Vial Compartment 212. Also shown is the Trash Chute lid 106 in a partly closed state, the Trash Chute lid 106 covering the Trash Chute that leads to the Waste Compartment (not shown) underneath the New Vial Compartment 204 and the Current Vial Compartment 212.

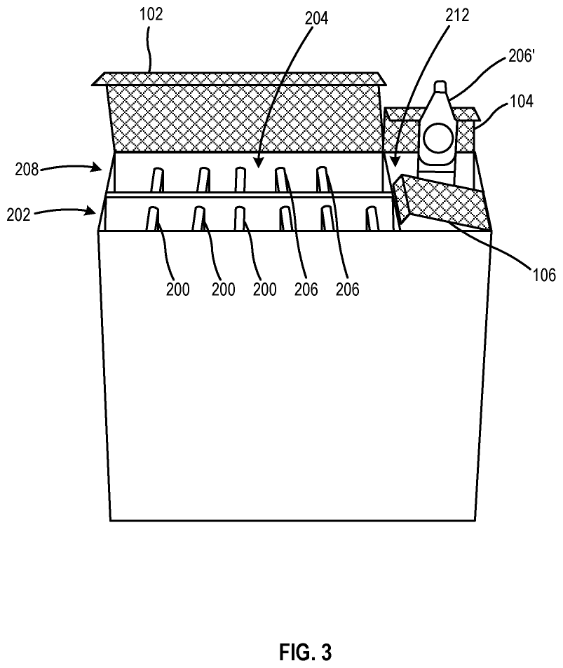

With reference to FIG. 3, the embodiment of FIG. 2 is again shown, where a new vial 206′ is missing from the set of new vials 206 in the second half 208 of the New Vial Compartment 204, and the partly used vial 206′ is shown entering the Current Vial Compartment 212. The Current Vial lid 104 is shown in a fully open state so that the partly used vial 206′ can be placed into the Current Vial Compartment 212.

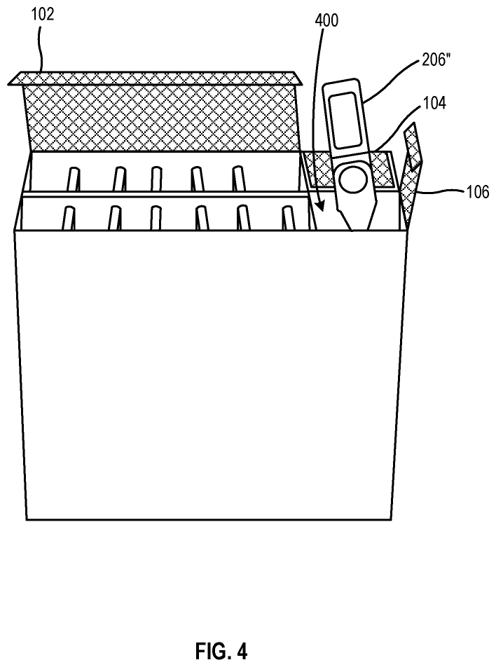

Referring to FIG. 4, the embodiment of FIG. 2 is shown with the Current Vial lid 104 in a closed state, also showing the partly used vial 206′ of FIG. 3 now shown as an empty vial 206″ entering the Trash Chute 400.

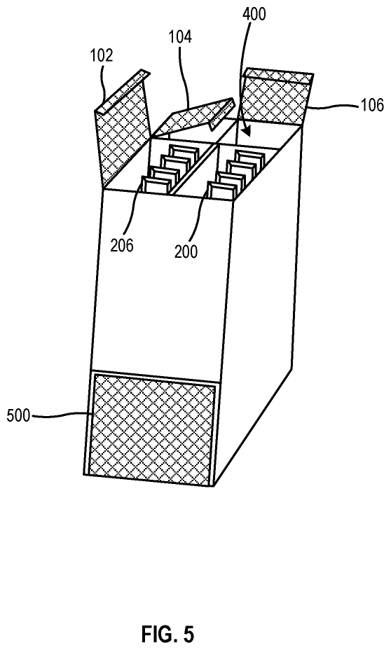

With reference to FIG. 5, the embodiment of FIG. 2 is again shown, showing only four of the new vials 200 and four of the new vials 206 still in the New Vial Compartment 204. The Current Vial Compartment 212 is shown empty with the Current Vial lid in a partly open state. Also, the top Trash Chute lid 106 is shown in an open state after the empty vial 206″ was dropped through the Trash Chute 400 and into the Waste Compartment (not shown). Also shown in a closed state is the side Waste Lid 500 of the Waste Compartment (not shown).

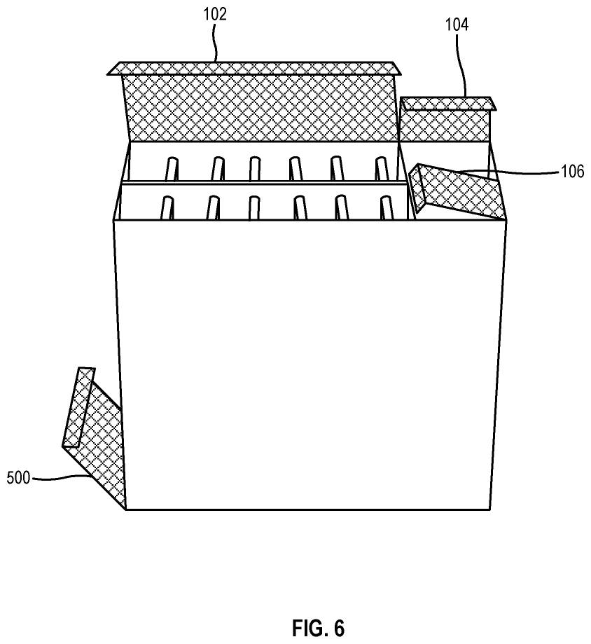

Referring to FIG. 6, the top Trash Chute lid 106 is shown in a partly closed state, while the side Waste Lid 500 of the Waste Compartment is shown in a partly open state. The lids 102 and 104 are shown in a mostly open state.

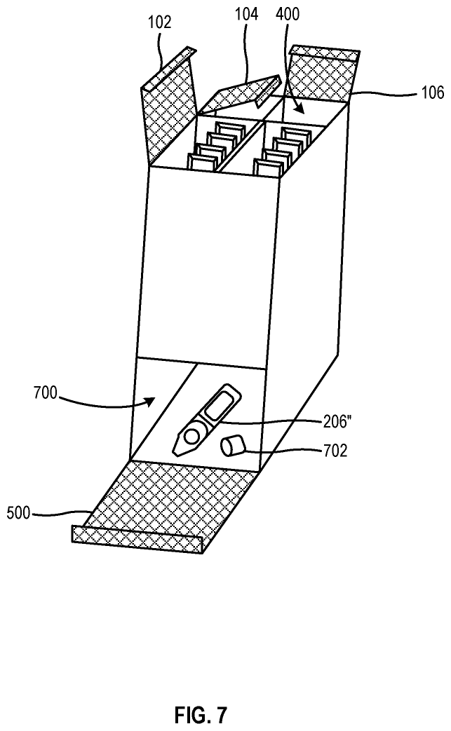

With reference to FIG. 7, the embodiment of FIG. 6 is shown with the top Trash Chute lid 106 in an open state, and the side Waste Lid 500 of the Waste Compartment 700 in a fully open state, revealing the empty vial 206″ along with its detached tab 702 resting on the floor of the Waste Compartment 700.

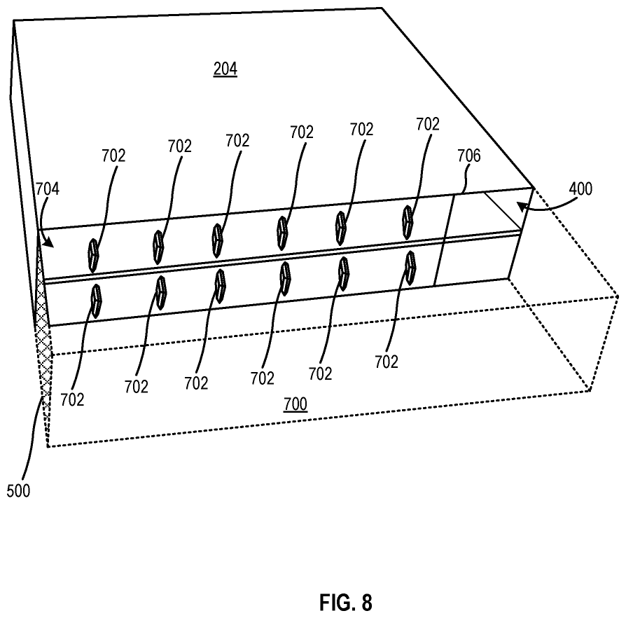

Referring to FIG. 8, a bottom perspective view of the embodiment of FIG. 7 is shown, revealing each tab 702 of a full set of new vials each inserted into a respective slot in the floor 704 of the New Vial Compartment 204, also showing the side Waste Lid 500 of the Waste Compartment 700 in a fully closed state, and showing the bottom opening 706 of the Trash Chute 400 that empties into the Waste Compartment 700.

Thus, the device 100 has three main Compartments:
1. The New Vial Compartment 204 accessed from the top via the New Lid 102, the New Vial Compartment 204 being divided in this embodiment into two sub-compartments 202 and 208 by a dividing wall 210, each sub-compartment for storing each of two different types of eyedrops.
2. The Current Vial Compartment 212 accessed from the top via Current Lid 104, the Current Vial Compartment 212 being for storing a single recently-opened vial 206′ to be used during that day.
3. The Waste Compartment 700 which is accessed from the top via the Trash Chute Lid 106, the Waste Compartment 700 being reached via the Trash Chute 400 for inserting used vials 206″ into the Waste Compartment, which is accessed from the side via the Waste Lid 500, which is opened when needed to empty the Waste Compartment 700.

Referring again to FIG. 2, the New Vial Compartment 204 is a space for NEW vials that is accessed from the top via the New Lid 102. The New Vial Compartment 204 houses unopened new eye drop vials 200 and 206 stored upside down, the tab (not shown in FIG. 2) of each vial 200, 206 inserted into a slot in the floor of the New Vial Compartment 204, as shown in FIG. 8. In the New Vial Compartment 204, there can be one row of one type of eye drop vials, or there can be more than one of these rows, as needed. The rows can be color-coded to help indicate different types of drops (e.g., drops with lower or higher viscosity for mild to severe dry eye symptoms, respectively). This New Vial Compartment 204 has a latching lid (called New Lid 102) that remains closed until opened as needed.

When a new eye drop vial is needed, the lid is unlatched and lifted, revealing a plurality of new vials. The new vials are stored upside down such that the tab of each new vial rests in a slot in the floor of the New Vial Compartment 204. To open a vial, the bottom of the vial (which extends upward) is grasped and twisted relative to the tab of the vial that rests in the slot, thereby opening the vial and separating the vial from the tab, which remains in the slot. Once opened, the tab top will fall down through the slot into the Waste Compartment 700 below the New Vial Compartment 204. Because the center of an eye drop vial must be squeezed to extract the eyedrop fluid, no loss of fluid will occur by storing the eye drop vial upside down and twisting it to open by separating the vial from the tab. The opened vial can then be removed from the New Vial Compartment 204, and an eye drop can be administered to the eye. Once the eye drop vial 206′ is removed, the New Lid 102 can be closed and latched.

Referring again to FIG. 3, the Current Vial Compartment 212 is a space for a CURRENT vial 206′ (a recently-opened partially used vial to be used that day), the space being accessible via the “Current Lid” 104. Once the eye drop has been administered using the vial, there may be some remaining fluid in the opened vial. This remaining fluid can be administered to the eye within 24 hours of opening the vial. The Current Vial Compartment 212 provides a space to store this “recently-opened” partially used vial. The Current Vial Compartment 212 has only enough space to store a single eye drop vial. The Current Vial Compartment 212 has a latching lid (Current Lid 104) that remains closed until needed. The Current Vial Compartment 212 can be color-coded, or otherwise marked to distinguish it as a “recently-opened vial to be used within 24 hours”. The Current Lid 104 can be opened throughout the day (within a 24 hour period) to retrieve the open vial 206′ to administer the remaining fluid to the eye as needed. At the end of the day, or as needed, the Current Lid 104 can be opened, and the used daily vial 206′ can be discarded by grasping the top of the vial so as to pick it up and then drop it into the Trash Chute 400 by opening the Trash Chute Lid 106 and then inserting the used vial into the Trash Chute 400 that empties directly into the Waste Compartment 700 below the New Vial Compartment 204. Note: the tab, once removed from the vial 206, does not need to be returned to the open vial 206′ to close it or re-seal it, because the vial 206′ needs to be squeezed in the center to extract the liquid. The Current Lid 104 will protect the opened vial 206′ that will be reused within 24 hours. Thus, there is no need to save the tabs after they are removed from the vials by twisting. Each tab can be dropped into the Trash Chute 400 after it has been separated to open the vial.

Referring again to FIGS. 7 and 8, the Waste Compartment 700 is a space for WASTE (used vials 206″ and tabs 702) accessed via the Trash Chute 400 that is covered by the Trash Chute Lid 106, the Waste Compartment 700 being emptied via the side Waste Lid 500. The Waste Compartment 700 has both a top opening Trash Chute Lid 106 and a side opening at the bottom via the Waste Lid 500. The Trash Chute Lid 106 of the trash chute 400 provides a place to insert a used eye drop vial 206″. Once inserted into Waste Compartment 700, the used vial 206″ slides down through the trash chute 400 and into the Waste Compartment 700 under the New Vial Compartment 204. The Waste Compartment 700 is a compartment that spans the entire width of the unit 100. The Waste Compartment 700 is configured to receive the empty vials 206″ inserted into the trash chute 400, and all of the removed tabs 702 from the opened vials 200 and 206. The Waste Lid 106 opens to provide access to the Waste Compartment 700 so that it can be emptied of all of the used vials 206″ and separated tabs 702. The Waste Lid 500 latches securely so when the unit is upright, the separated tabs 702 and empty vials 206″ are secured inside the Waste Compartment 700. When it is time to empty the Waste Compartment 700, the Waste Lid 500 can be unlatched, allowing it to open so that the separated tabs 702 and the empty vials 206″ can fall out into a trash container. Other modifications and implementations will occur to those skilled in the art without departing from the spirit and the scope of the invention as claimed. Accordingly, the above description is not intended to limit the invention, except as indicated in the following claims.

What is claimed is:

1. Apparatus for management of single-use eye drop vials, each single-use eye drop vial being sealed with a twist-off tab, the apparatus comprising:
a first new vial storage compartment configured to store a first plurality of new vials, the first new vial storage compartment having a top lid, and a bottom panel having a plurality of slots, each slot configured to receive a twist-off tab of a new vial when the new vial is inserted therein;
a second new vial storage compartment configured to store a second plurality of new vials, the second new vial storage compartment having a top lid, and a bottom panel having a plurality of slots, each slot configured to receive a twist-off tab of a new vial when the new vial is inserted therein; and
a trash storage compartment located below both the first and second storage compartments, the trash storage compartment configured to receive a plurality of twist-off tabs and a plurality of used vials, the trash storage compartment having a top lid for receiving used vials, and a bottom lid for emptying the trash storage compartment.

2. The apparatus of claim 1, further comprising:
an opened-vial storage compartment configured to store a single opened partially used vial, the single opened partially used vial for use within 24 hours of opening.

3. The apparatus of claim 2, wherein the opened-vial storage compartment has a separately operable latching lid.

4. The apparatus of claim 2, wherein the opened-vial storage compartment is configured to appear distinct from both the first and second new vial storage compartments.

5. The apparatus of claim 2, wherein the opened-vial storage compartment is configured to appear distinct from both the first and second new vial storage compartments by imparting a different color to the opened-vial storage compartment.

6. The apparatus of claim 1, wherein the first new vial storage compartment and the second new vial storage compartment are configured to appear distinct such that a user can separately store two different types of eye drops, one type in each of the first and second new vial storage compartments.

7. The apparatus of claim 6, wherein the first and second new vial storage compartments are configured to appear distinct by imparting a different color to each new vial storage compartment.

8. The apparatus of claim 1, wherein each lid is configured to be latched open and latched closed.

9. The apparatus of claim 1, wherein the first storage compartment top lid, and the second storage compartment top lid are continuous.

10. The apparatus of claim 1, wherein the first storage compartment top lid, the second storage compartment top lid, and the used vial storage compartment top lid are all continuous with each other.

11. The apparatus of claim 1, wherein the first new vial storage compartment is configured to store a first plurality of new vials in an inverted vertical orientation, each new vial having a twist-off tab underneath the new vial.

12. The apparatus of claim 1, wherein the trash storage compartment is configured to receive a plurality of used vials either vertically or horizontally.

13. The apparatus of claim 1, wherein the apparatus is made from a material that is light-weight so as to enhance portability.

14. The apparatus of claim 1, wherein the bottom panel has a plurality of slots, each slot configured to receive a twist-off tab of a new vial when the new vial is inserted therein, and each slot is configured to resist turning of an inserted twist-off tab, while also allowing the twist-off tab once separated from the new vial to pass down through the slot into the trash storage compartment.

15. The apparatus of claim 14, wherein the trash storage compartment has a side-opening lid that is latched.

16. The apparatus of claim 14, wherein the trash storage compartment has a bottom-opening lid that is latched.

17. The apparatus of claim 1, wherein the trash storage compartment includes a divider configured to separate the twist-off tabs and the used vials.

18. The apparatus of claim 17, wherein the trash storage compartment has two independently openable lids, a first lid for inserting used vials and twist-off tabs, and a second lid for removing used vials and twist-off tabs.

19. Apparatus for management of single-use eye drop vials, each single-use eye drop vial being sealed with a twist-off tab, the apparatus comprising:
a first new vial storage compartment configured to store a first plurality of new vials, the first new vial storage compartment having a top lid, and a bottom panel having a plurality of slots, each slot configured to receive a twist-off tab of a new vial when the new vial is inserted therein;
a second new vial storage compartment, adjacent to the first new vial storage compartment, the second new vial storage compartment configured to store a second plurality of new vials, the second new vial storage compartment having a top lid, and a bottom panel having a plurality of slots, each slot configured to receive a twist-off tab of a new vial when the new vial is inserted therein;
an opened-vial storage compartment, adjacent to at least one of the first new vial storage compartment and the second new vial storage compartment, the opened-vial storage compartment configured to store a single opened partially used vial, the single opened partially used vial for use within 24 hours of opening; and
a trash storage compartment located below both the first and second storage compartments, the trash storage compartment configured to receive a plurality of twist-off tabs and a plurality of used vials, the trash storage compartment having a top lid for receiving used vials, and a bottom lid for emptying the trash storage compartment.

20. The apparatus of claim 19, wherein the opened-vial storage compartment has a separately operable latching lid, and the opened-vial storage compartment is configured to appear distinct from both the first new vial storage compartment and the second new vial storage compartment.
